(19) United States
(12) Patent Application Publication
(10) Pub. No.: US 2020/0148657 A1
(43) Pub. Date: May 14, 2020

(54) PROCESS FOR PREPARING 5-HYDROXYMETHYLFURFURAL

(71) Applicant: FAR EASTERN NEW CENTURY CORPORATION, Taipei City (TW)

(72) Inventors: Ying-Jen Chen, Taipei City (TW); Yi-Fen Yang, Taipei City (TW); Fa-Chen Chi, Taipei City (TW); Ruey-Fen Liao, Taipei City (TW); Chien-Hung Chou, Taipei City (TW)

(73) Assignee: FAR EASTERN NEW CENTURY CORPORATION, Taipei City (TW)

(21) Appl. No.: 16/413,180

(22) Filed: May 15, 2019

(30) Foreign Application Priority Data
Nov. 14, 2018 (TW) 107140425
Nov. 27, 2018 (TW) 107142330

(51) Int. Cl. C07D 307/50

(52) U.S. Cl. CPC C07D 307/50

(57) ABSTRACT

A process for preparing 5-hydroxymethylfurfural includes the steps of: subjecting a glucose-containing material to a catalytic reaction in the presence of an isomerase enzyme to form an aqueous saccharide solution containing fructose and glucose, converting the fructose into 5-hydroxymethylfurfural via a first dehydration reaction, and converting the glucose into 5-hydroxymethylfurfural via a second dehydration reaction.

### CROSS-REFERENCE TO RELATED APPLICATION

This application claims priority of Taiwanese Application No. 107140425, filed on Nov. 14, 2018 and priority of Taiwanese Application No. 107142330, filed on Nov. 27, 2018.

### FIELD

The disclosure relates to a process for preparing 5-hydroxymethylfurfural, and more particularly to a process for preparing 5-hydroxymethylfurfural from a glucose-containing material.

### BACKGROUND

5-hydroxymethylfurfural (referred hereinafter to as 5-HMF) is an important intermediate in the production of medicine and biomaterials, and is widely applied in chemical, biopharmaceutical, agricultural, and other industries.

5-HMF can be chemically converted into various hydrogenated derivatives such as 2,5-bis(hydroxymethyl) furan, 2,5-bis(hydroxymethyl) tetrahydrofuran, 2,5-dimethylfuran, 2,5-dimethyltetrahydrofuran, 1,2,6-hexane-triol, and 1,6-hexanediol. In addition, 5-HMF can be oxidized to obtain 2,5-furandicarboxylic acid. 2,5-furandicarboxylic acid can be subjected to a polymerization reaction with ethylene glycol to form polyethylene 2,5-furandicarboxylate, which is a polyester material having gas barrier property.

Mass-scale production of 5-HMF in tons is usually carried out by using fructose as a raw material. Fructose is subjected to a dehydration reaction in the presence of an acidic solution to obtain 5-HMF. However, fructose is relatively costly as compared to glucose and cellulose. Therefore, glucose and/or cellulose is used in place of fructose in some studies for production of 5-HMF.

U.S. Patent Publication No. 2014/0349351 A1 discloses a process for producing 5-HMF. The process comprises the steps of: a) providing an aqueous solution comprising fructose and, optionally, glucose and/or mannose; b) optionally contacting the solution with glucose isomerase enzyme (E.C.5.3.1.5) which converts glucose to fructose, and/or mannose isomerase enzyme (E.C. 5.3.1.7) which converts mannose to fructose; c) combining the solution with at least one organic solvent as well as an acid catalyst and/or a salt to provide a reaction mixture, wherein the reaction mixture forms a single-phase system at standard conditions of 20° C. and 1 atm absolute pressure; and d) heating the reaction mixture for a time period that is sufficient to allow dehydration of fructose so as to obtain 5-HMF as a resulting product. Since glucose contained in the aqueous solution provided in step (a) cannot be fully converted into fructose in step (b), the reaction mixture in steps (c) and (d) still contains residual glucose, which cannot be converted into 5-HMF under reaction conditions of steps (c) and (d), and thus, the yield of 5-HMF is unsatisfactory.

In the process for producing 5-HMF from a mixture of glucose and fructose, fructose is converted into 5-HMF at a relatively high rate. Since 5-HMF is a thermally unstable compound, during the conversion of glucose into 5-HMF at an elevated temperature for a long time period, 5-HMF formed from fructose may be polymerized to form humins or hydrolyzed to form levulinic acid, resulting in undesirable decrease in a total yield of 5-HMF.

U.S. Pat. No. 9260402B2 discloses a process for preparing 5-HMF, in which fructose or glucose may be used in a starting mixture for preparing 5-HMF. The process for preparing 5-HMF is implemented in the presence of an ionic liquid and a metal halide catalyst, and a purification means such as distillation is required to obtain 5-HMF. Since the ionic liquid is a costly solvent, the process for preparing 5-HMF incurs a high production cost.

It is also disclosed in other prior art that preparation of 5-HMF from glucose can be implemented in the presence of catalysts such as phosphoric acid (H₃PO₄), Nb₂O₅, meso-TiO₂, phosphoric acid treated Nb₂O₅, CZS (SO₄²⁻/ZrO₂), AlCl₃, B(OH)₃, Ag₃PW₁₂O₄₀, SnCl₄, CrCl₃, Al(O-iPr)₃, Al(Et)₃, and the like. However, a relatively larger amount of such catalysts is required in said other prior art, and the yield obtained thereby is usually lower than 65%. Specifically, when an aqueous glucose solution with a concentration of 30 wt % is used for preparing 5-HMF in the presence of Ag₃PW₁₂O₄₀ as a catalyst, the yield obtained thereby is 64%. However, the amount of the catalyst used is as high as 13.3 wt %. Therefore, the production cost of 5-HMF cannot be effectively reduced.

In view of the aforesaid, it is desirable to develop a process for preparing 5-HMF which uses a relatively cheap material such as glucose or cellulose, and which can overcome the aforesaid shortcomings of the prior art for achieving the purpose of commercialization.

### SUMMARY

An object of the disclosure is to provide a process for preparing 5-hydroxymethylfurfural which can reduce production cost and effectively enhance productivity.

According to the disclosure, there is provided a process for preparing 5-hydroxymethylfurfural, comprising the steps of:

(a) subjecting a glucose-containing material to a catalytic reaction in the presence of an isomerase enzyme to form an aqueous saccharide solution containing fructose and glucose;

(b) mixing the aqueous saccharide solution with an inorganic acid and an organic solvent to form a liquid mixture;

(c) subjecting the liquid mixture to a first dehydration reaction under heating to convert the fructose into 5-hydroxymethylfurfural to obtain a reaction mixture containing 5-hydroxymethylfurfural and the glucose; and

(d) subjecting the reaction mixture to a second dehydration reaction in the presence of a metal halide to convert the glucose into 5-hydroxymethylfurfural.

In the process for preparing 5-hydroxymethylfurfural according to the disclosure, a glucose-containing material is used, which is a relatively cheap material compared to fructose. An aqueous saccharide solution containing fructose and glucose is formed from the glucose-containing material via a catalytic reaction in the presence of an isomerase enzyme. The aqueous saccharide solution is then subjected to a first dehydration reaction and a second dehydration reaction in sequence to obtain 5-hydroxymethylfurfural. Specifically, in the first dehydration reaction, fructose contained in the aqueous saccharide solution is first converted into 5-hydroxymethylfurfural in the presence of an inorganic acid. Glucose contained in the aqueous saccharide solution is not reacted in the first dehydration reaction and remains in a reaction mixture formed after the first dehydration reaction. That is, the reaction mixture includes 5-hydroxymethylfurfural, glucose, an inorganic acid, an organic solvent, and water. In the second dehydration reaction, glucose in the reaction mixture is converted into 5-hydroxymethylfurfural in the presence of a metal halide serving as a catalyst. Fructose and glucose contained in the aqueous saccharide solution formed from the glucose-containing material can be converted into 5-hydroxymethylfurfural more effectively via the first dehydration reaction and the second dehydration reaction, respectively. 5-hydroxymethylfurfural formed in the first dehydration reaction is not further reacted in the second dehydration reaction. Therefore, the total yield of 5-hydroxymethylfurfural obtained by the process according to the disclosure can be enhanced significantly.

### DETAILED DESCRIPTION

A process for preparing 5-hydroxymethylfurfural comprises the steps of:

(a) subjecting a glucose-containing material to a catalytic reaction in the presence of an isomerase enzyme to form an aqueous saccharide solution containing fructose and glucose;

(b) mixing the aqueous saccharide solution with an inorganic acid and an organic solvent to form a liquid mixture;

(c) subjecting the liquid mixture to a first dehydration reaction under heating to convert the fructose into 5-hydroxymethylfurfural to obtain a reaction mixture containing 5-hydroxymethylfurfural and the glucose; and

(d) subjecting the reaction mixture to a second dehydration reaction in the presence of a metal halide to convert the glucose into 5-hydroxymethylfurfural.

### Glucose-Containing Material:

The glucose-containing material used in step (a) can be glucose, any material containing glucose, or any material that will produce glucose via a reaction. In certain embodiments, the glucose-containing material can be glucose, a hydrolysis product of starch, a hydrolysis product of sucrose, a hydrolysis product of cellulose, a hydrolysis product of hemicellulose, a hydrolysis product of cellulose biomass, or combinations thereof. The cellulose biomass can be any biomass material containing cellulose, and can be derived from a single material or a combination of two or more different materials. Examples of the cellulose biomass include, but are not limited to, bioenergy crops, agricultural residues, municipal solid waste, industrial solid waste, paper sludge, yard waste, waste wood, and forestry waste. In certain embodiments, the cellulose biomass is selected from the group consisting of plant, paper (specifically, waste paper), and a combination thereof. Examples of the plant include, but are not limited to, miscanthus, cork, hardwood, corncob, crop residue (e.g., corn husk), corn stalk, grass, wheat stalk, barley stalk, hay, rice straw, switchgrass, bagasse, sorghum plant material, corn plant material, grain powders, tree, branch, root, leaf, wood chips, bush, vegetable, fruit, and flower. The glucose-containing material used in following illustrated examples includes a commercially available aqueous glucose solution and a hydrolysis product of rice straw.

When glucose is used as the glucose-containing material, the glucose is not required to undergo any pretreatment or hydrolysis reaction, and only needs to be prepared into an aqueous glucose solution in a suitable concentration. The aqueous glucose solution thus obtained can be used directly in the process for preparing 5-hydroxymethylfurfural according to the disclosure. When biomass such as starch, sucrose, cellulose, hemicellulose, cellulose biomass, or combinations thereof is used for preparing the glucose-containing material, it is pre-treated with any means well known in the art to form the glucose-containing material. For example, the biomass can be subjected to a dilute acid treatment, a solid-liquid separation, a steam explosion or the like, followed by a hydrolysis reaction in the presence of a suitable hydrolase to obtain a hydrolysis product. Specifically, when the cellulose biomass is used for preparing the glucose-containing material, it is treated by mixing with a dilute acid solution to form a liquid mixture, subjecting the liquid mixture to a degradation reaction in an autoclave at an elevated temperature (for example, 120° C.) to form a reaction mixture containing hemicellulose, subjecting the reaction mixture to a solid-liquid separation to obtain a solid portion, subjecting the solid portion to a steam explosion in a steam explosion system to obtain an acid-treated residue, and subjecting the acid-treated residue to a hydrolysis reaction in the presence of an enzyme mixture to obtain a hydrolysis product of cellulose biomass serving as the glucose-containing material. The enzyme mixture can be a mixture of cellulase and hemicellulase, for example, Cellic® CTec2 commercially available from Novozymes A/S.

The operation procedure and the reaction conditions for the hydrolysis reaction can be adjusted suitably according to relevant technique and knowledge in the art. In certain embodiments, the hydrolysis reaction is implemented under stirring in the presence of the enzyme mixture at a temperature ranging from 50° C. to 60° C. for a period ranging from 48 hours to 96 hours. In certain embodiments, the cellulase or the enzyme mixture containing the cellulase is used in an amount ranging from 12 FPU/g glucose to 15 FPU/g glucose. In the following examples, the hydrolysis reaction is implemented in the presence of the enzyme mixture containing cellulase and hemicellulase at a temperature of 50° C. for a period of 72 hours.

In step (a) of the process for preparing 5-hydroxymethylfurfural according to the disclosure, the glucose-containing material is subjected to a catalytic reaction in the presence of an isomerase enzyme to form an aqueous saccharide solution containing fructose and glucose. The isomerase enzyme can be any glucose isomerase used in the art, for example, E.C.5.3.1.5 or the like. The operation procedure, the equipment, and the reaction conditions for implementing the catalytic reaction in step (a) can be adjusted suitably according to relevant technique and knowledge in the art. In certain embodiments, the catalytic reaction is implemented at a temperature ranging from 45° C. to 70° C. In certain embodiments, the catalytic reaction is implemented at a temperature ranging from 50° C. to 60° C. In certain embodiments, the catalytic reaction is implemented at a temperature ranging from 50° C. to 55° C. In certain embodiments, the catalytic reaction is implemented for a period ranging from 36 hours to 72 hours. The catalytic reaction can be implemented by directly adding the isomerase enzyme into the glucose-containing material or by filling the isomerase enzyme into a column, followed by passing the glucose-containing material through the column. In the following examples, the isomerase enzyme is filled into a column, and the glucose-containing material is then circulated through the column to implement the catalytic reaction.

In certain embodiments, the aqueous saccharide solution formed in step (a) contains glucose in an amount ranging from 50 wt % to 56 wt % and fructose in an amount ranging from 44 wt % to 50 wt %.

In step (b), the aqueous saccharide solution is mixed with an inorganic acid and an organic solvent to forma liquid mixture. The temperature for implementing step (b) can be adjusted suitably according to relevant technique and knowledge in the art. In certain embodiments, the temperature for implementing step (b) is in a range from 0° C. to 40° C.

The inorganic acid suitable for the process according to the disclosure can be any inorganic acid suitable for a dehydration reaction of glucose, and examples thereof include, but are not limited to, hydrochloric acid, sulfuric acid, phosphoric acid, boric acid, nitric acid, and combinations thereof. The inorganic acid used in the following examples includes hydrochloric acid and sulfuric acid.

The organic solvent suitable for the process according to the disclosure can be any organic solvent for the dehydration reaction of glucose, and examples thereof include, but are not limited to, acetone, tetrahydrofuran, butanol, γ-valerolactone, and combinations thereof. The organic solvent used in the following examples includes acetone and tetrahydrofuran.

In certain embodiments, step (b) includes the sub-steps of: (b1) mixing the aqueous saccharide solution with an inorganic acid to form an acidic solution which has a concentration of the inorganic acid in a range from 0.03 M to 0.15 M; and (b2) mixing the acidic solution with the organic solvent to form the liquid mixture. In certain embodiments, the concentration of the inorganic acid is in a range from 0.05 M to 0.1 M. The concentration of the inorganic acid in the following examples is 0.065 M, 0.07 M, or 0.090 M.

In step (c), the liquid mixture is subjected to a first dehydration reaction under heating to convert the fructose into 5-hydroxymethylfurfural to obtain a reaction mixture containing 5-hydroxymethylfurfural and the glucose. The temperature for the first dehydration reaction can be adjusted suitably according to the types and the amounts of the components in the liquid mixture. In certain embodiments, the first dehydration reaction is implemented at a temperature ranging from 130° C. to 180° C. In certain embodiments, the temperature for the first dehydration reaction is in a range from 140° C. to 175° C. In certain embodiments, the temperature for the first dehydration reaction is in a range from 150° C. to 170° C. In the following examples, the first dehydration reaction is implemented at a temperature of 150° C.

In step (d), the reaction mixture is subjected to a second dehydration reaction in the presence of a metal halide to convert the glucose into 5-hydroxymethylfurfural. In certain embodiments, the second dehydration reaction is implemented at a temperature ranging from 130° C. to 180° C. In certain embodiments, the temperature for the second dehydration reaction is in a range from 140° C. to 175° C. In certain embodiments, the temperature for the second dehydration reaction is in a range from 150° C. to 170° C. In the following examples, the second dehydration reaction is implemented at a temperature of 150° C.

The metal halide for the second dehydration reaction can be any metal halide catalyst which is well known for converting glucose into 5-hydroxymethylfurfural. In certain embodiments, the metal halide can be represented by a formula of MX₃, wherein M is selected from the group consisting of Al, In, and Fe, and X is selected from the group consisting of Cl, Br, and I. The metal halide used in the following examples includes AlCl₃ and InCl₃. In certain embodiments, the metal halide is used in an amount ranging from 2 mmol to 5 mmol.

It should be noted that although the aqueous saccharide solution formed in step (a) contains both fructose and glucose, 5-hydroxymethylfurfural obtained from the first dehydration reaction in step (c) is not further reacted in the second dehydration reaction in step (d). Therefore, the total yield of 5-hydroxymethylfurfural obtained by the process according to the disclosure can be enhanced significantly.

Examples of the disclosure will be described hereinafter. It is to be understood that these examples are exemplary and explanatory and should not be construed as a limitation to the disclosure.

### Preparation Examples 1 to 5: Preparation of Glucose-Containing Material

Rice straw purchased from Hong-Yuan Agricultural Products (Taiwan) was chopped and was then ground using a grinder into a powdery material. The powdery material was mixed with an aqueous sulfuric acid solution having a concentration shown in Table 1 to forma liquid mixture. The liquid mixture was added into an autoclave (100 L, manufacturer: Lucky Seven Industrial Co., Ltd., Taiwan), followed by a degradation reaction using the reaction parameters shown in Table 1 to form a reaction mixture containing hemicellulose.

The reaction mixture was placed into a filter bag (Manufacturer: Yi-Chang Filter Cloth Co., Ltd., Taiwan; Model: PP 60350S) having a pore size of 37 μm, followed by a solid-liquid separation using a vertical press machine (Manufacturer: RESI Corp., Taiwan) at a pressure of 8 MPa to obtain a solid portion and a liquid portion.

The solid portion was placed into a steam explosion system (Manufacturer: Lucky Seven Industrial Co., Ltd., Taiwan), followed by a steam explosion treatment which was implemented by introducing steam into the steam explosion system, heating the steam explosion system using the reaction parameters shown in Table 1, and quickly reducing the pressure within the steam explosion system to 1 atm. An acid-treated residue collected after the steam explosion treatment was dried, and was analyzed using a Hitachi Chromaster 5100 HPLC system to determine the contents of glucose and xylose in the acid-treated residue. The results are shown in Table 1.

The acid-treated residue was mixed with an enzyme mixture (i.e., Cellic® CTec2 manufactured by Novozymes A/S), followed by a hydrolysis reaction at 50° C. under stirring at 150 rpm for 72 hours to obtain a rice straw hydrolysis product.

TABLE 1

| | Concentration | | | Composition of | |
|---|---|---|---|---|---|
| | of aqueous | Temperature and | Temperature | acid-treated | |
| | sulfuric acid | time for | and time for | residue after | Yield of |
| Preparation | solution | degradation | steam | drying | glucose |
| Example | (wt %) | reaction | explosion | (wt %) | (%)* |
| 1 | 1 | 121° C./3 hr | 200° C./3 hr | Glucose = 54.7 | 82 |
| | | | | Xylose = 2.3 | |
| 2 | 2 | 121° C./2 hr | 190° C./5 hr | Glucose = 60 | 76 |
| | | | | Xylose = 5 | |
| 3 | 2 | 121° C./2 hr | 200° C./1 hr | Glucose = 56 | 74 |
| | | | | Xylose = 6 | |
| 4 | 3 | 121° C./2 hr | 190° C./5 hr | Glucose = 59 | 81 |
| | | | | Xylose = 2 | |
| 5 | 3 | 121° C./2 hr | 200° C./1 hr | Glucose = 57 | 83 |
| | | | | Xylose = 4 | |

*Yield of glucose was calculated on a basis of an original composition of the rice straw, and represents glucose content in the acid-treated residue after drying.

### Examples 1 to 5

Each of Examples 1 to 5 was implemented according to the following procedure using the materials and the reaction parameters shown in Table 3.

Glucose isomerase enzyme (E.C.5.3.1.5, 30 g) was filled into a column (diameter: 1.5 cm, height: 40 cm), followed by filling a commercially available aqueous glucose solution (purchased from Echo Chemical Co., Ltd., concentration: 80.0 g/L, referred to as “Commercially-available” in Table 3) or a rice straw hydrolysis product of Preparation Example 4 (referred to as “Prep. Ex. 4” in Table 3) into the column using a peristaltic pump, and implementing a catalytic reaction by circulation in the presence of the glucose isomerase enzyme to form an aqueous saccharide solution containing fructose and glucose. A weight ratio of fructose and glucose in the aqueous saccharide solution was analyzed using a DIONEX Ultimate 3000 HPLC system equipped with an Aminex® HPX-87H column (Manufacturer: Bio-Rad Laboratories Inc., Model: 1250140, 7.8×300 ml). Injection volume for the analysis was 20 μl and the temperature of the column was maintained at 65° C. An aqueous sulfuric acid solution having a concentration of 5 mM was used as a mobile phase at a flow rate of 0.5 ml/min. The DIONEX Ultimate 3000 HPLC system was equipped with a refractive index detector with a detection temperature of 45° C. Retention times for fructose and glucose were 9.5 min and 10.5 min, respectively. The analysis results are shown in Table 3.

The aqueous saccharide solution was mixed with an inorganic acid to form an acidic solution. The acidic solution (150 ml) was then mixed with an organic solvent (750 ml) to form a liquid mixture.

The liquid mixture was subjected to a first dehydration reaction at a temperature of 150° C. for a first reaction period shown in Table 3 to convert fructose into 5-hydroxymethylfurfural to obtain a reaction mixture containing 5-hydroxymethylfurfural and glucose.

A metal halide was added into the reaction mixture, followed by a second dehydration reaction at a temperature of 150° C. for a second reaction period shown in Table 3 to convert glucose into 5-hydroxymethylfurfural so as to obtain a reaction product.

Analysis of 5-hydroxymethylfurfural Yield:

The analysis was implemented using a DIONEX Ultimate 3000 HPLC system equipped with a SunFire™ C18 column (5 μm, 4.6×250 ml). Injection volume for the analysis was 10 μl and the temperature of the column was maintained at 25° C. A mobile phase and flow rates of the analysis are shown in Table 2. An ultraviolent absorption spectrometer was used as a detector at 254 nm with a reference wavelength of 400 nm and a band width of 10 nm. The retention time for 5-hydroxymethylfurfural is 6.9 min. The analysis results are shown in Table 3.

TABLE 2

| | | Time (min) | | | | | | | | | |
|---|---|---|---|---|---|---|---|---|---|---|---|
| | | 0.0 | 3.0 | 4.0 | 11.0 | 11.1 | 14.1 | 14.2 | 22.2 | 22.3 | 24.3 |
| Mobile | Acetonitrile | 1 | 1 | 3 | 5 | 10 | 10 | 1 | 1 | 1 | 1 |
| phase | 0.05% aqueous | 9 | 9 | 7 | 5 | 0 | 0 | 9 | 9 | 9 | 9 |
| | phosphoric | | | | | | | | | | |
| | acid solution | | | | | | | | | | |

| | | | | | | | | | | |
|---|---|---|---|---|---|---|---|---|---|---|
| Flow rate (ml/min) | 1.0 | 1.0 | 1.0 | 1.0 | 1.0 | 1.0 | 1.2 | 1.2 | 1.0 | 1.0 |

TABLE 3

| Example | 1 | 2 | 3 | 4 | 5 |
|---|---|---|---|---|---|
| Glucose-containing | Commercially- | Commercially- | Commercially- | Prep. Ex. | Prep. Ex. |
| material | available | available | available | 4 | 4 |
| Weight ratio of | 51/49 | 51/49 | 51/49 | 51/49 | 51/49 |
| glucose/fructose in | | | | | |
| an aqueous | | | | | |
| saccharide solution | | | | | |
| Inorganic acid | Hydro- | Hydro- | Hydro- | Sulfuric | Sulfuric |
| | chloric acid | chloric acid | chloric acid | acid | acid |
| Concentration of | 0.090 | 0.090 | 0.090 | 0.070 | 0.065 |
| inorganic acid in an | | | | | |
| acidic solution (M) | | | | | |
| Organic solvent | Acetone | THF | Acetone | Acetone | Acetone |
| Type of metal halide | AlCl₃ | AlCl₃ | InCl₃ | AlCl₃ | AlCl₃ |
| Amount of metal | 0.500 | 0.500 | 0.829 | 0.497 | 0.497 |
| halide g (mmol) | (3.75) | (3.75) | (3.75) | (3.73) | (3.73) |
| First reaction | 30 | 30 | 30 | 30 | 30 |
| period (min) | | | | | |
| Second reaction | 30 | 30 | 30 | 25 | 30 |
| period (min) | | | | | |
| Yield of | 68 | 61 | 59 | 73 | 81 |
| 5-hydroxymethyl- | | | | | |
| furfural(%)^{a} | | | | | |

^{a}on a basis of an initial total amount of fructose and glucose obtained by analysis via HPLC

### Comparative Examples 1-1 to 1-7

In each of Comparative Examples 1-1 to 1-7, an aqueous glucose solution was used to implement a single dehydration reaction directly. Specifically, the materials and the reaction parameters shown in Table 4 were used. A commercially available aqueous glucose solution (120 ml, 80.0 g/L) was mixed with an inorganic acid to form an acidic solution. The acidic solution (150 ml) was then mixed with an organic solvent (750 ml) to form a liquid mixture.

AlCl₃ (0.500 g, 3.75 mmol) was added into the liquid mixture, followed by a single dehydration reaction at a temperature for a reaction period shown in Table 4 to obtain a reaction product. The reaction product was cooled to a room temperature, followed by filtration and condensation under reduced pressure to obtain an aqueous solution containing 5-hydroxymethylfurfural. The yield of 5-hydroxymethylfurfural of each of Comparative Examples 1-1 to 1-7 was analyzed according to the analysis method in Examples 1 to 5. The results are shown in Table 4.

TABLE 4

| Comp. Ex. | 1-1 | 1-2 | 1-3 | 1-4 | 1-5 | 1-6 | 1-7 |
|---|---|---|---|---|---|---|---|
| Inorganic | Hydro- | Hydro- | Hydro- | Hydro- | Hydro- | Sulfuric | Hydro- |
| acid | chloric | chloric | chloric | chloric | chloric | acid | chloric |
| | acid | acid | acid | acid | acid | | acid |
| Concentration | 0.007 | 0.007 | 0.007 | 0.007 | 0.090 | 0.090 | 0.095 |
| of inorganic | | | | | | | |
| acid in an | | | | | | | |
| acidic | | | | | | | |
| solution (M) | | | | | | | |
| Organic | Acetone | Acetone | Acetone | THF | Acetone | Acetone | Acetone |
| solvent | | | | | | | |
| Reaction | 130 | 140 | 150 | 150 | 150 | 150 | 150 |
| temperature | | | | | | | |
| (° C.) | | | | | | | |
| Reaction | 20 | 20 | 20 | 20 | 20 | 30 | 20 |
| period (min) | | | | | | | |
| Yield of | 14 | 37 | 52 | 46 | 56 | 54 | 42 |
| 5-hydroxy- | | | | | | | |
| methyl- | | | | | | | |
| furfural (%) | | | | | | | |

### Comparative Examples 2-1 to 2-3

In each of Comparative Examples 2-1 to 2-3, glucose was converted into an aqueous saccharide solution containing fructose and glucose, and the aqueous saccharide solution was then subjected to a single dehydration reaction directly. Specifically, glucose isomerase enzyme (E.C.5.3.1.5, 30 g) was filled into a column (diameter: 1.5 cm, height: 40 cm), followed by filling a commercially available aqueous glucose solution (purchased from Echo Chemical Co., Ltd., concentration: 80.0 g/L) into the column using a peristaltic pump, and implementing a catalytic reaction by circulation in the presence of the glucose isomerase enzyme at 60° C. for 48 hours to form an aqueous saccharide solution containing fructose and glucose. A weight ratio of fructose and glucose in the aqueous saccharide solution was analyzed according the analysis method in Examples 1 to 5. The analysis results are shown in Table 5.

The aqueous saccharide solution was mixed with an inorganic acid (0.090 M) to form an acidic solution. The acidic solution (150 ml) was then mixed with acetone (750 ml) to form a liquid mixture.

AlCl₃ was added in an amount shown in Table 5 into the liquid mixture, followed by a single dehydration reaction at a temperature of 150° C. fora reaction period shown in Table 5 to obtain a reaction product. The reaction product was cooled to a room temperature, followed by filtration and condensation under reduced pressure to obtain an aqueous solution containing 5-hydroxymethylfurfural. The yield of 5-hydroxymethylfurfural of each of Comparative Examples 2-1 to 2-3 was analyzed according to the analysis method in Examples 1 to 5. The results are shown in Table 5.

### Comparative Examples 3 to 5

Each of Comparative Examples 3 to 5 was implemented according to the procedure of each of Examples 1 to 5 using the materials and reaction parameters shown in Table 5, except that the second dehydration reaction in each of Examples 1 to 5 was not implemented in Comparative Examples 3 to 5.

TABLE 5

| Comp. Ex. | 2-1 | 2-2 | 2-3 | 3 | 4 | 5 |
|---|---|---|---|---|---|---|
| Glucose-containing | Commercially- | Commercially- | Commercially- | Commercially- | Prep. Ex. | Prep. Ex. |
| material | available | available | available | available | 4 | 4 |
| Weight ratio of | 51/49 | 51/49 | 51/49 | 51/49 | 51/49 | 51/49 |
| glucose/fructose | | | | | | |
| in an aqueous | | | | | | |
| saccharide solution | | | | | | |
| Inorganic acid | Hydro- | Hydro- | Sulfuric | Hydro- | Sulfuric | Sulfuric |
| | chloric | chloric | acid | chloric | acid | acid |
| | acid | acid | | acid | | |
| Concentration of | 0.090 | 0.090 | 0.090 | 0.090 | 0.070 | 0.065 |
| inorganic acid | | | | | | |
| in an acidic | | | | | | |
| solution (M) | | | | | | |
| Organic solvent | Acetone | Acetone | Acetone | Acetone | Acetone | Acetone |
| Reaction | 20 | 60 | 20 | 30 | 30 | 30 |
| period (min) | | | | | | |
| Type of metal halide | AlCl₃ | AlCl₃ | AlCl₃ | None | None | None |
| Amount of metal | 0.50 | 0.50 | 0.497 | 0 | 0 | 0 |
| halide g (mmol) | (3.75) | (3.75) | (3.73) | | | |
| Yield of | 60 | 52 | 53 | 37 | 54 | 53 |
| 5-hydroxy- | | | | | | |
| methylfurfural (%) | | | | | | |

As shown in Table 3, the yield of 5-hydroxymethylfurfural in Examples 1 to 5 is in a range from 59% to 81%. Specifically, in Examples 1, 2, 4, and 5 in which AlCl₃ is used as the metal halide for the second dehydration reaction, the yield of 5-hydroxymethfurfural is further enhanced to a range from 61% to 81%. More specifically, in Examples 4 and 5 in which AlCl₃ is used as the metal halide for the second dehydration reaction and sulfuric acid is used as the inorganic acid, the yield of 5-hydroxymethfurfural is as high as 81%. It is demonstrated that the yield of 5-hydroxymethfurfural can be enhanced effectively by the process according to the disclosure, in which a glucose-containing material is converted into an aqueous saccharide solution containing fructose and glucose, and the aqueous saccharide solution is then subjected to a first dehydration reaction and a second dehydration reaction in sequence to obtain 5-hydroxymethylfurfural.

A comparison of Example 1 and Comparative Example 2-2 is shown in Table 6.

TABLE 6

| | First | Second | Yield of |
|---|---|---|---|
| | reaction | reaction | 5-hydroxymeth- |
| | period (min) | period (min) | furfural (%) |
| Example 1 | 30 | 30 | 68 |

| | | |
|---|---|---|
| Comparative | 60 | 52 |
| Example 2-2 | (a reaction period for a | |
| | single dehydration | |
| | reaction of fructose and | |
| | glucose simultaneously) | |

As shown in Table 6, in Comparative Example 2-2, in which fructose and glucose are subjected to a dehydration reaction simultaneously, the yield of the thus obtained 5-hydroxymethfurfural is only 52%. In Example 1, in which fructose and glucose are subjected to a first dehydration reaction and a second dehydration, respectively, the yield of the thus obtained 5-hydroxymethfurfural is as high as 68%.

A comparison of Example 1 and Comparative Examples 1-7 and 3 is shown in Table 7.

TABLE 7

| | | Concen- | | | | |
|---|---|---|---|---|---|---|
| | | tration | | | | |
| | Weight | of | | | | |
| | ratio of | hydro- | Amount | | | |
| | glucose/ | chloric | of | | | Yield of |
| | fructose in | acid in | metal | First | Second | 5-hydroxy- |
| | an aqueous | an acidic | halide | reaction | reaction | methyl- |
| | saccharide | solution | g | period | period | furfural |
| | solution | (M) | (mmol) | (min) | (min) | (%) |
| Ex. 1 | 51%/49% | 0.090 | 0.500 | 30 | 30 | 68 |
| | | | (3.75) | | | |

| | | | | | |
|---|---|---|---|---|---|
| Comp. | 100/0 | 0.095 | 0.500 | 20 | 42 |
| Ex. 1-7 | (without a | | (3.75) | (a single | |
| | catalytic | | | dehydration | |
| | reaction | | | reaction | |
| | in the | | | of glucose) | |
| | presence | | | | |
| | of an | | | | |
| | isomerase | | | | |
| | enzyme) | | | | |
| Comp. | 51%/49% | 0.090 | none | 30 | 37 |
| Ex. 3 | | | | (a single | |
| | | | | dehydration | |
| | | | | reaction of | |
| | | | | fructose) | |

As shown in Table 7, in Comparative Example 1-7, in which a single dehydration reaction of glucose is implemented, the yield of the thus obtained 5-hydroxymethylfurfural is only 42%. In Comparative Example 3, in which a single dehydration reaction of fructose is implemented without a further dehydration reaction of glucose, the yield of the thus obtained 5-hydroxymethylfurfural is only 37%. In Example 1, in which fructose and glucose are subjected to a first dehydration reaction and a second dehydration, respectively, the yield of the thus obtained 5-hydroxymethylfurfural is as high as 68%. It is demonstrated that according to the process of the disclosure, in which fructose and glucose are subjected to the first dehydration reaction and the second dehydration, respectively, the yield of the thus obtained 5-hydroxymethylfurfural can be enhanced effectively and the possibility of 5-hydroxymethylfurfural undergoing further reaction (for example, polymerization to form humins or hydrolysis to form levulinic acid) is reduced significantly.

A comparison of Examples 4 and 5 to Comparative Example 2-3 is shown in Table 8.

TABLE 8

| | Concentration | | | |
|---|---|---|---|---|
| | of sulfuric | First | Second | |
| | acid in an | reaction | reaction | Yield of |
| | acidic | period | period | 5-hydroxymethyl- |
| | solution (M) | (min) | (min) | furfural (%) |
| Example 4 | 0.070 | 30 | 25 | 73 |
| Example 5 | 0.065 | 30 | 30 | 81 |

| | | | |
|---|---|---|---|
| Comparative | 0.090 | 20 | 53 |
| Example 2-3 | | Simultaneous | |
| | | dehydration | |
| | | reaction of | |
| | | fructose and | |
| | | glucose | |

As shown in Table 8, in Comparative Example 2-3, in which fructose and glucose contained in an aqueous saccharide solution are subjected to a dehydration reaction simultaneously, the yield of the thus obtained 5-hydroxymethylfurfural is only 53%. In Examples 4 and 5, in which fructose and glucose are subjected to a first dehydration reaction and a second dehydration, respectively, the yields of the thus obtained 5-hydroxymethylfurfural are as high as 73% and 81%, respectively.

A comparison of Examples 4 and 5 and Comparative Example 1-6, and Comparative Examples 4 and 5 is shown in Table 9.

TABLE 9

| | Weight | Concen- | | | | |
|---|---|---|---|---|---|---|
| | ratio of | tration | Amount | | | Yield |
| | glucose/ | of | of | | | of 5- |
| | fructose in | sulfuric | metal | First | Second | hydroxy- |
| | an aqueous | acid in | halide | reaction | reaction | methyl- |
| | saccharide | an acidic | g | period | period | furfural |
| | solution | solution (M) | (mmol) | (min) | (min) | (%) |
| Ex. 4 | 51%/49% | 0.070 | 0.497 | 30 | 25 | 73 |
| | | | (3.73) | | | |
| Ex. 5 | 51%/49% | 0.065 | 0.497 | 30 | 30 | 81 |
| | | | (3.73) | | | |

| | | | | | |
|---|---|---|---|---|---|
| Comp. | 100/0 | 0.090 | 0.500 | 30 | 54 |
| Ex. | (without a | | (3.75) | (a single | |
| 1-6 | catalytic | | | dehydration | |
| | reaction | | | reaction | |
| | in the | | | of glucose) | |
| | presence | | | | |
| | of an | | | | |
| | isomerase | | | | |
| | enzyme) | | | | |
| Comp. | 51%/49% | 0.070 | None | 30 | 54 |
| Ex. 4 | | | | (a single | |
| | | | | dehydration | |
| | | | | reaction of | |
| | | | | fructose) | |
| Comp. | 51%/49% | 0.065 | None | 30 | 53 |
| Ex. 5 | | | | (a single | |
| | | | | dehydration | |
| | | | | reaction of | |
| | | | | fructose) | |

As shown in Table 9, in Comparative Example 1-6, in which a single dehydration reaction of glucose is implemented, the yield of the thus obtained 5-hydroxymethylfurfural is only 54%. In Comparative Examples 4 and 5, in which a single dehydration reaction of fructose is implemented, the yields of the thus obtained 5-hydroxymethylfurfural are only 54% and 53%, respectively. In Examples 4 and 5, the yields of the thus obtained 5-hydroxymethylfurfural are as high as 73% and 81%, respectively.

In summary, according to the process for preparing 5-hydroxymethylfurfural of the disclosure, in which fructose and glucose are subjected to a first dehydration reaction and a second dehydration, respectively, 5-hydroxymethylfurfural can be obtained in an enhanced yield from a relatively cheap glucose-containing material. In addition, the possibility of 5-hydroxymethylfurfural undergoing further reaction (for example, polymerization to form humins or hydrolysis to form levulinic acid) can be reduced significantly.

In the description above, for the purposes of explanation, numerous specific details have been set forth in order to provide a thorough understanding of the embodiment(s). It will be apparent, however, to one skilled in the art, that one or more other embodiments maybe practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A process for preparing 5-hydroxymethylfurfural, comprising the steps of:
(a) subjecting a glucose-containing material to a catalytic reaction in the presence of an isomerase enzyme to form an aqueous saccharide solution containing fructose and glucose;
(b) mixing the aqueous saccharide solution with an inorganic acid and an organic solvent to form a liquid mixture;
(c) subjecting the liquid mixture to a first dehydration reaction under heating to convert the fructose into 5-hydroxymethylfurfural to obtain a reaction mixture containing 5-hydroxymethylfurfural and the glucose; and
(d) subjecting the reaction mixture to a second dehydration reaction in the presence of a metal halide to convert the glucose into 5-hydroxymethylfurfural.

2. The process according to claim 1, wherein in step (a), the glucose-containing material is selected from the group consisting of glucose, a hydrolysis product of starch, a hydrolysis product of sucrose, a hydrolysis product of cellulose, a hydrolysis product of hemicellulose, a hydrolysis product of cellulose biomass, and combinations thereof.

3. The process according to claim 2, wherein the glucose-containing material is the hydrolysis product of cellulose biomass, and the cellulose biomass is selected from the group consisting of plant, paper, and a combination thereof.

4. The process according to claim 1, wherein in step (a), the catalytic reaction is implemented at a temperature ranging from 45° C. to 70° C.

5. The process according to claim 1, wherein in step (b), the inorganic acid is selected from the group consisting of hydrochloric acid, sulfuric acid, phosphoric acid, boric acid, nitric acid, and combinations thereof.

6. The process according to claim 1, wherein in step (b), the organic solvent is selected from the group consisting of acetone, tetrahydrofuran, butanol, γ-valerolactone, and combinations thereof.

7. The process according to claim 1, wherein step (b) includes the sub-steps of:
(b1) mixing the aqueous saccharide solution with an inorganic acid to form an acidic solution which has a concentration of the inorganic acid in a range from 0.03 M to 0.15 M; and
(b2) mixing the acidic solution with the organic solvent to form the liquid mixture.

8. The process according to claim 1, wherein in step (c), the first dehydration reaction is implemented at a temperature ranging from 130° C. to 180°.

9. The process according to claim 1, wherein the metal halide is represented by a formula of MX₃, wherein M is selected from the group consisting of Al, In, and Fe, and X is selected from the group consisting of Cl, Br, and I.

10. The process according to claim 1, wherein in step (d), the metal halide is used in an amount ranging from 2 mmol to 5 mmol.

11. The process according to claim 1, wherein in step (d), the second dehydration reaction is implemented at a temperature ranging from 130° C. to 180°.
